(19) United States
(12) Patent Application Publication
(10) Pub. No.: US 2003/0194421 A1
(43) Pub. Date: Oct. 16, 2003

(54) Treatment of uveitis

(75) Inventor: Pierre E. Signore, Vancouver (CA)

Correspondence Address: SEED INTELLECTUAL PROPERTY LAW GROUP PLLC, 701 FIFTH AVE, SUITE 6300, SEATTLE, WA, 98104-7092, US

(73) Assignee: Angiotech Pharmaceuticals, Inc., Vancouver (CA)

(21) Appl. No.: 10/331,126

(22) Filed: Dec. 27, 2002

Related U.S. Application Data
(60) Provisional application No. 60/344,009, filed on Dec. 28, 2001.

(51) Int. Cl. A61K 31/4745; A61K 31/337

(52) U.S. Cl. 424/426; 514/449; 514/283; 424/427

(57) ABSTRACT

Compositions, methods and devices are provided for treating and/or preventing uveitis.

### CROSS-REFERENCE TO RELATED APPLICATION

This application claims the benefit of U.S. Provisional Patent Application No. 60/344,009, filed Dec. 28, 2001, where this provisional application is incorporated herein by reference in its entirety.

### BACKGROUND OF THE INVENTION

1. Field of the Invention

The present invention relates generally to pharmaceutical compositions, methods, and devices, and more specifically, to methods for treating uveitis.

2. Description of the Related Art

Uveitis is a major cause of visual loss in the Western World. It is responsible for about 10% of the severe visual handicap in the United States. (Nussenblatt et al. 1996; Ann NY Acad Sci 778: 325-337). The disease affects 35,000 patients in the US and about 100,000 worldwide (CIBC World Market, Equity Research, 2001). Complications associated with intraoccular inflammation (uveitis) include posterior synechia, cataract, glaucoma and retinal edema (Smith et al., Immunology and Cell Biology 76: 497-512, 1998).

A wide variety of infective agents can cause uveitis. When an infective etiology has been diagnosed, an appropriate antimicrobial drug is given to cure the disease. However, the etiology of uveitis remains elusive in the majority of cases. The only treatment option left is to control the inflammatory symptoms. In such cases, corticosteroids are the gold standard as suppressors of inflammation in the eye. Anterior uveitis often responds to local steroid treatment with eye drops. However, drops do not usually provide therapeutic levels of steroids in the posterior part of the eye for the treatment of posterior uveitis or panuveitis. Perioccular injections are then indicated. They can be given subconjunctivally or beneath Tenon's capsule.

Systemic treatments with steroids are indicated when local injections fail but many of the most severe cases of uveitis do not respond to high dose systemic corticosteroid therapy. In addition, the side effects of systemic therapies can be devastating. They include hypertension, hyperglycemia, peptic ulceration, cushingoid feature, osteoporosis, growth limitation, myopathy, psychosis and susceptibility to infection. Finally, local and systemic steroid therapies also have sight-threatening side effects such as glaucoma, cataract and susceptibility to eye infection.

Several other compounds have been investigated to replace corticosteroids but none has succeeded in clinical trials. Non-steroidal anti-inflammatory drugs have proved disappointing in the treatment of eye disease. Other agents such as cyclosporin, antifolates (methotrexate), and antipurines (6-mercaptopurine, azathioprine) have shown a poor efficacy/toxicity ratio when given systemically. Newer immunosuppressive agents such as Tacrolimus, Sirolimus and mycophelonate mofetil are also being investigated but they have serious side effects (Anglade and Whitcup, Drugs 49:213-223, 1995). Therefore, there exists a need for a means and a method to treat inflammatory disease of the eye.

Thus, there is a need in the art for compositions and methods for treating uveitis that overcomes the difficulties associated with prior treatments. The present invention discloses such compositions and methods, and further, provides other, related advantages.

### BRIEF SUMMARY OF THE INVENTION

Briefly stated, the present invention provides compositions and methods for treating uveitis. The present invention involves administering antimicrotubule agents to patients to treat or prevent uveitis. In one embodiment of the invention antimicrotubule agents are administered systemically. In another embodiment, treatment is local by eye drops, iontophoresis, sonophoresis, or periocular injections. In another embodiment of the present invention, antimicrotubule treatment is intraoccular by injection or surgical insertion of a controlled release formulation. During cataract surgery the diseased, opaque intraoccular lens is removed and replaced by a clear synthetic lens. Cataract surgery is often followed by inflammatory complications. In another embodiment of the present invention, treatment is by insertion of an antimicrotubule agent-releasing intraoccular lens during cataract surgery.

Within other aspects of the invention, methods are provided for treating or preventing uveitis, comprising administering to a patient a anti-microtubule agent. Within certain embodiments, the anti-microtubule agent is paclitaxel, or an analogue or derivative thereof. Within other embodiments, the anti-microtubule agent is a topoisomerase inhibitor such as camptothecin, a vinca alkaloid such as vinblastine or vincristine, a nitrogen mustard, or, a podophyllotoxin. Within various embodiments, the anti-microtubule agent further comprises a polymer. Within further embodiments of the invention, the anti-microtubule agent is released directly into the eye (e.g., from an intraoccular lens or implant, or, by intraoccular or periocular injection into the eye). Within yet other embodiments, the anti-microtubule agent is administered systemically (e.g., in a micellar or liposomal carrier), or topically to the eye (e.g., by eye drops).

Within related aspects of the invention, devices are provided comprising an intraoccular lens which release an anti-microtubule into the eye. As noted above, the anti-microtubule agent may be released directly from the lens, or, from a composition (e.g., containing a polymer) that is coated onto all or a portion of the lens. Representative examples of anti-microtubule agents which can be released in this regard include paclitaxel, and analogues and derives thereof, topoisomerase inhibitors such as camptothecin, vinka alkaloids such as vinblastine or vincristine, nitrogen mustards, or, podophyllotoxins. Within preferred embodiments of the invention, the intraoccular lens is sterilized prior to implant.

These and other aspects of the present invention will become evident upon reference to the following detailed description and attached drawings. In addition, various references are set forth herein which describe in more detail certain procedures or compositions (e.g., compounds, proteins, vectors, and their generation, etc.), and are therefore incorporated by reference in their entirety. When PCT applications are referred to it is also understood that the underlying or cited U.S. applications are also incorporated by reference herein in their entirety.

### DETAILED DESCRIPTION OF THE INVENTION

Prior to setting forth the invention, it may be helpful to an understanding thereof to set forth definitions of certain terms that will be used hereinafter.

“Uveitis” refers to intraoccular conditions associated with acute or chronic inflammation. In acute inflammation, the main infiltrating cells are polymorphonuclear neutrophils and macrophages accompanied by edema, vascular dilation and congestion. Tissue damage can result in necrosis. In contrast, the main infiltrating cells in chronic inflammation are lymphocytes and macrophages with exudate, vascular congestion and obstruction. Inflammation is further categorized into granulomatous or non-granulomatous depending on the presence of epithelioid and giant cells surrounded by lymphocytes and macrophages. (Chan and Li, British J. of Ophthalmology 82:91-96, 1998).

Inflammatory processes are usually associated with the capillary network. Since the uvea is the most vascular structure of the eye, histological signs of inflammation are found in the uvea even when the cause is located in adjacent structures. Additionally, ocular structures other than the uveal tract including the sclera, retina and vitreous humour may also be affected by the inflammatory response and are included among uveitic entities. Three main types of uveitis may be distinguished by the location of inflammatory reaction. Anterior uveitis is confined to structures anterior to the lens (cornea, iris and ciliary body). Intermediate uveitis involves structures just posterior to the lens. Posterior uveitis is ocular inflammation in the choroid, retina and vitreous. Panuveitis includes anterior and posterior segments of the eye. Pathologically, uveitis is classified as “endogenous” when the ocular inflammation results from an inflammatory, immune or metabolic disease or “exogenous” when the ocular inflammation is a sequel of traumatic or surgical perforation of the eye.

“Anti-microtubule Agents” should be understood to include any protein, peptide, chemical, or other molecule which impairs the function of microtubules, for example, through the prevention or stabilization of polymerization. A wide variety of methods may be utilized to determine the anti-microtubule activity of a particular compound, including for example, assays described by Smith et al. (Cancer Lett 79(2):213-219, 1994) and Mooberry et al., (Cancer Lett. 96(2):261-266, 1995).

As noted above, the present invention provides methods for treating or preventing inflammatory disease of the eye, comprising the step of delivering to the eye an anti-microtubule agent. Discussed in more detail below are (I) Anti-Microtubule Agents; (II) Anti-Microtubule Agent Compositions and Formulations; and (III) Clinical Applications.

### I. Anti-Microtubule Agents

Briefly, a wide variety of anti-microtubule agents can be utilized within the context of the present invention, either with or without a carrier (e.g., a polymer or ointment; see section II below).

Representative examples of such agents include taxanes (e.g., paclitaxel (discussed in more detail below) and docetaxel) (Schiff et al., Nature 277: 665-667, 1979; Long and Fairchild, Cancer Research 54: 4355-4361, 1994; Ringel and Horwitz, J. Natl. Cancer Inst. 83(4): 288-291, 1991; Pazdur et al, Cancer Treat. Rev. 19(4): 351-386, 1993), campothecin, mitoxantrone, eleutherobin (e.g., U.S. Pat. No. 5,473,057), sarcodictyins (including sarcodictyin A), epothilones A and B (Bollag et al., Cancer Research 55: 2325-2333, 1995), discodermolide (ter Haar et al., Biochemistry 35: 243-250, 1996), deuterium oxide (D₂O) (James and Lefebvre, Genetics 130(2): 305-314, 1992; Sollott et al., J. Clin. Invest. 95: 1869-1876, 1995), hexylene glycol (2-methyl-2,4-pentanediol) (Oka et al., Cell Struct. Funct. 16(2): 125-134, 1991), tubercidin (7-deazaadenosine) (Mooberry et al., Cancer Lett. 96(2): 261-266, 1995), LY290181 (2-amino-4-(3-pyridyl)-4H-naphtho(1,2-b)pyran-3-cardonitrile) (Panda et al., J. Biol. Chem. 272(12): 7681-7687, 1997; Wood et al., Mol. Pharmacol. 52(3): 437-444, 1997), aluminum fluoride (Song et al., J. Cell. Sci. Suppl. 14: 147-150, 1991), ethylene glycol bis-(succinimidylsuccinate) (Caplow and Shanks, J. Biol. Chem. 265(15): 8935-8941, 1990), glycine ethyl ester (Mejillano et al., Biochemistry 31(13): 3478-3483, 1992), nocodazole (Ding et al., J. Exp. Med 171(3): 715-727, 1990; Dotti et al., J. Cell Sci. Suppl. 15: 75-84, 1991; Oka et al., Cell Struct. Funct. 16(2): 125-134, 1991; Weimer et al., J. Cell. Biol. 136(1), 71-80, 1997), cytochalasin B (Illinger et al., Biol. Cell 73(2-3): 131-138, 1991), colchicine and CI 980 (Allen et al., Am. J. Physiol. 261(4 Pt. 1): L315-L321, 1991; Ding et al., J. Exp. Med. 171(3): 715-727, 1990; Gonzalez et al., Exp. Cell. Res. 192(1): 10-15, 1991; Stargell et al., Mol. Cell. Biol. 12(4): 1443-1450, 1992; Garcia et al., Antican. Drugs 6(4): 533-544, 1995), colcemid (Barlow et al., Cell. Motil. Cytoskeleton 19(1): 9-17, 1991; Meschini et al., J. Microsc. 176(Pt. 3): 204-210, 1994; Oka et al., Cell Struct. Funct. 16(2): 125-134, 1991), podophyllotoxin (Ding et al., J. Exp. Med. 171(3): 715-727, 1990), benomyl (Hardwick et al., J. Cell. Biol. 131(3): 709-720, 1995; Shero et al., Genes Dev. 5(4): 549-560, 1991), oryzalin (Stargell et al., Mol. Cell. Biol. 12(4): 1443-1450, 1992), majusculamide C (Moore, J. Ind. Microbiol. 16(2): 134-143, 1996), demecolcine (Van Dolah and Ramsdell, J. Cell. Physiol. 166(1): 49-56, 1996; Wiemer et al., J. Cell. Biol. 136(1): 71-80, 1997), methyl-2-benzimidazolecarbamate (MBC) (Brown et al., J. Cell. Biol. 123(2): 387-403, 1993), LY195448 (Barlow & Cabral, Cell Motil. Cytoskel. 19: 9-17, 1991), subtilisin (Saoudi et al., J. Cell Sci. 108: 357-367, 1995), 1069C85 (Raynaud et al., Cancer Chemother Pharmacol. 35: 169-173, 1994), steganacin (Hamel, Med. Res. Rev. 16(2): 207-231, 1996), combretastatins (Hamel, Med Res. Rev. 16(2): 207-231, 1996), curacins (Hamel, Med. Res. Rev. 16(2): 207-231, 1996), estradiol (Aizu-Yokata et al., Carcinogen. 15(9): 1875-1879, 1994), 2-methoxyestradiol (Hamel, Med. Res. Rev. 16(2): 207-231, 1996), flavanols (Hamel, Med. Res. Rev. 16(2): 207-231, 1996), rotenone (Hamel, Med Res. Rev. 16(2): 207-231, 1996), griseofulvin (Hamel, Med Res. Rev. 16(2): 207-231, 1996), vinca alkaloids, including vinblastine and vincristine (Ding et al., J. Exp. Med 171(3): 715-727, 1990; Dirk et al., Neurochem. Res. 15(11): 1135-1139, 1990; Hamel, Med. Res. Rev. 16(2): 207-231, 1996; Illinger et al., Biol. Cell 73(2-3): 131-138, 1991; Wiemer et al., J. Cell. Biol. 136(1): 71-80, 1997), maytansinoids and ansamitocins (Hamel, Med Res. Rev. 16(2): 207-231, 1996), rhizoxin (Hamel, Med. Res. Rev. 16(2): 207-231, 1996), phomopsin A (Hamel, Med. Res. Rev. 16(2): 207-231, 1996), ustiloxins (Hamel, Med. Res. Rev. 16(2): 207-231, 1996), dolastatin 10 (Hamel, Med Res. Rev. 16(2): 207-231, 1996), dolastatin 15 (Hamel, Med. Res. Rev. 16(2): 207-231, 1996), halichondrins and halistatins (Hamel, Med. Res. Rev. 16(2): 207-231, 1996), spongistatins (Hamel, Med. Res. Rev. 16(2): 207-231, 1996), cryptophycins (Hamel, Med. Res. Rev. 16(2): 207-231, 1996), rhazinilam (Hamel, Med. Res. Rev. 16(2): 207-231, 1996), betaine (Hashimoto et al., Zool. Sci. 1: 195-204, 1984), taurine (Hashimoto et al., Zool. Sci. 1: 195-204, 1984), isethionate (Hashimoto et al., Zool. Sci. 1: 195-204, 1984), HO-221 (Ando et al., Cancer Chemother Pharmacol. 37: 63-69, 1995), adociasulfate-2 (Sakowicz et al., Science 280: 292-295, 1998), estramustine (Panda et al., Proc. Natl. Acad. Sci. USA 94: 10560-10564, 1997), monoclonal anti-idiotypic antibodies (Leu et al., Proc. Natl. Acad. Sci. USA 91(22): 10690-10694, 1994), microtubule assembly promoting protein (taxol-like protein, TALP) (Hwang et al., Biochem. Biophys. Res. Commun. 208(3): 1174-1180, 1995), cell swelling induced by hypotonic (190 mosmol/L) conditions, insulin (100 mmol/L) or glutamine (10 mmol/L) (Haussinger et al., Biochem. Cell. Biol. 72(1-2): 12-19, 1994), dynein binding (Ohba et al., Biochim. Biophys. Acta 1158(3): 323-332, 1993), gibberelin (Mita and Shibaoka, Protoplasma 119(1/2): 100-109, 1984), XCHOI (kinesin-like protein) (Yonetani et al., Mol. Biol. Cell 7(suppl): 211 A, 1996), lysophosphatidic acid (Cook et al., Mol. Biol. Cell 6(suppl): 260A, 1995), lithium ion (Bhattacharyya and Wolff, Biochem. Biophys. Res. Commun. 73(2): 383-390, 1976), plant cell wall components (e.g., poly-L-lysine and extensin) (Akashi et al., Planta 182(3): 363-369, 1990), glycerol buffers (Schilstra et al., Biochem. J. 277(Pt. 3): 839-847, 1991; Farrell and Keates, Biochem. Cell. Biol. 68(11): 1256-1261, 1990; Lopez et al., J. Cell. Biochem. 43(3): 281-291, 1990), Triton X-100 microtubule stabilizing buffer (Brown et al., J. Cell Sci. 104(Pt. 2): 339-352, 1993; Safiejko-Mroczka and Bell, J. Histochem. Cytochem. 44(6): 641-656, 1996), microtubule associated proteins (e.g., MAP2, MAP4, tau, big tau, ensconsin, elongation factor-1-alpha (EF-1α) and E-MAP-115) (Burgess et al., Cell Motil. Cytoskeleton 20(4): 289-300, 1991; Saoudi et al., J. Cell. Sci. 108(Pt. 1): 357-367, 1995; Bulinski and Bossler, J. Cell. Sci. 107(Pt. 10): 2839-2849, 1994; Ookata et al., J. Cell Biol. 128(5): 849-862, 1995; Boyne et al., J. Comp. Neurol 358(2): 279-293, 1995; Ferreira and Caceres, J. Neurosci. 11(2): 392-400, 1991; Thurston et al., Chromosoma 105(1):20-30, 1996; Wang et al., Brain Res. Mol. Brain Res. 38(2): 200-208, 1996; Moore and Cyr, Mol. Biol. Cell 7(suppl): 221-A, 1996; Masson and Kreis, J. Cell Biol. 123(2), 357-371, 1993), cellular entities (e.g., histone H1, myelin basic protein and kinetochores) (Saoudi et al., J. Cell. Sci. 108(Pt. 1): 357-367, 1995; Simerly et al., J. Cell Biol. 111(4): 1491-1504, 1990), endogenous microtubular structures (e.g., axonemal structures, plugs and GTP caps) (Dye et al., Cell Motil. Cytoskeleton 21(3): 171-186, 1992; Azhar and Murphy, Cell Motil. Cytoskeleton 15(3): 156-161, 1990; Walker et al., J. Cell Biol. 114(1): 73-81, 1991; Drechsel and Kirschner, Curr. Biol. 4(12): 1053-1061, 1994), stable tubule only polypeptide (e.g., STOP145 and STOP220) (Pirollet et al., Biochim. Biophys. Acta 1160(1): 113-119, 1992; Pirollet et al., Biochemistry 31(37): 8849-8855, 1992; Bosc et al., Proc. Natl. Acad. Sci. USA 93(5): 2125-2130, 1996; Margolis et al., EMBO J. 9(12): 4095-4102, 1990) and tension from mitotic forces (Nicklas and Ward, J. Cell Biol. 126(5): 1241-1253, 1994), as well as any analogues and derivatives of any of the above. Such compounds can act by either depolymerizing microtubules (e.g., colchicine and vinblastine), or by stabilizing microtubule formation (e.g., paclitaxel).

A. Paclitaxel, Analogues and Derivatives

Within one preferred embodiment of the invention, the anti-microtubule agent is paclitaxel, a compound which disrupts mitosis (M-phase) by binding to tubulin to form abnormal mitotic spindles or an analogue or derivative thereof. Briefly, paclitaxel is a highly derivatized diterpenoid (Wani et al., J. Am. Chem. Soc. 93:2325, 1971) which has been obtained from the harvested and dried bark of Taxus brevifolia (Pacific Yew) and Taxomyces Andreanae and Endophytic Fungus of the Pacific Yew (Stierle et al., Science 60:214-216, 1993). “Paclitaxel” (which should be understood herein to include formulations, prodrugs, analogues and derivatives such as, for example, TAXOL®, TAXOTERE® or docetaxel, 10-desacetyl analogues of paclitaxel and 3′N-desbenzoyl-3′N-t-butoxy carbonyl analogues of paclitaxel) may be readily prepared utilizing techniques known to those skilled in the art (see, e.g., Schiff et al., Nature 277:665-667, 1979; Long and Fairchild, Cancer Research 54:4355-4361, 1994; Ringel and Horwitz, J. Nat'l Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402—from Taxus brevifolia).

Representative examples of paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol (2′-and/or 7-O-ester derivatives), (2′-and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-7-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl) taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogs with modified phenylisoserine side chains, taxotere, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin); and other taxane analogues and derivatives, including 14-beta-hydroxy-10 deacetybaccatin III, debenzoyl-2-acyl paclitaxel derivatives, benzoate paclitaxel derivatives, phosphonooxy and carbonate paclitaxel derivatives, sulfonated 2′-acryloyltaxol; sulfonated 2′-O-acyl acid paclitaxel derivatives, 18-site-substituted paclitaxel derivatives, chlorinated paclitaxel analogues, C4 methoxy ether paclitaxel derivatives, sulfenamide taxane derivatives, brominated paclitaxel analogues, Girard taxane derivatives, nitrophenyl paclitaxel, 10-deacetylated substituted paclitaxel derivatives, 14-beta-hydroxy-10 deacetylbaccatin III taxane derivatives, C7 taxane derivatives, C10 taxane derivatives, 2-debenzoyl-2-acyl taxane derivatives, 2-debenzoyl and -2-acyl paclitaxel derivatives, taxane and baccatin III analogs bearing new C2 and C4 functional groups, n-acyl paclitaxel analogues, 10-deacetylbaccatin III and 7-protected-10-deacetylbaccatin III derivatives from 10-deacetyl taxol A, 10-deacetyl taxol B, and 10-deacetyl taxol, benzoate derivatives of taxol, 2-aroyl-4-acyl paclitaxel analogues, orthro-ester paclitaxel analogues, 2-aroyl-4-acyl paclitaxel analogues and 1-deoxy paclitaxel and 1-deoxy paclitaxel analogues.

In one aspect, the Anti-microtubule agent is a taxane having the formula (C1):

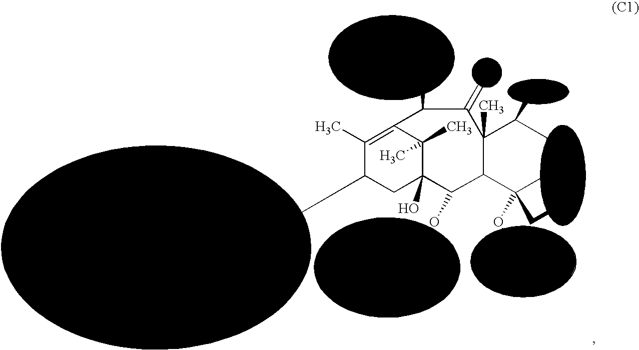

where the gray-highlighted portions may be substituted and the non-highlighted portion is the taxane core. A side-chain (labeled “A” in the diagram ) is desirably present in order for the compound to have good activity as a Anti-microtubule agent. Examples of compounds having this structure include paclitaxel (Merck Index entry 7117), docetaxol (Taxotere, Merck Index entry 3458), and 3′-desphenyl-3′-(4-ntirophenyl)-N-debenzoyl-N-(t-butoxycarbonyl)-10-deacetyltaxol.

In one aspect, suitable taxanes such as paclitaxel and its analogs and derivatives are disclosed in U.S. Pat. No. 5,440,056 as having the structure (C2):

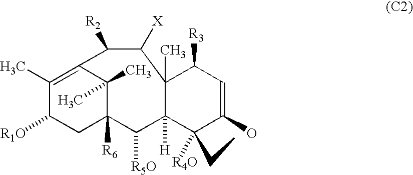

wherein X may be oxygen (paclitaxel), hydrogen (9-deoxy derivatives), thioacyl, or dihydroxyl precursors; R₁ is selected from paclitaxel or taxotere side chains or alkanoyl of the formula (C3)

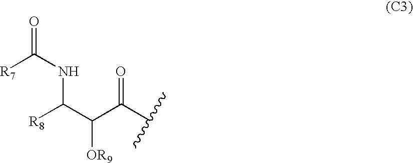

wherein R₇ is selected from hydrogen, alkyl, phenyl, alkoxy, amino, phenoxy (substituted or unsubstituted); R₈ is selected from hydrogen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidyalkanoyloxy; R₃ is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In one aspect, the paclitaxel analogs and derivatives useful as Anti-microtubule agents in the present invention are disclosed in PCT International Patent Application No. WO 93/10076. As disclosed in this publication, the analog or derivative should have a side chain attached to the taxane nucleus at C₁₃, as shown in the structure below (formula C4), in order to confer antitumor activity to the taxane.

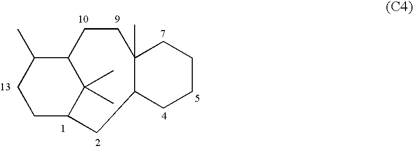

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, 10. As well, an oxetane ring may be attached at carbons 4 and 5. As well, an oxirane ring may be attached to the carbon labeled 4.

In one aspect, the taxane-based Anti-microtubule agent useful in the present invention is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

Taxanes in general, and paclitaxel in particular, are considered to function as anti-microtubule agents by stabilizing microtubules.

B. Vinca Alkaloids

In another aspect, the Anti-microtubule agent is a Vinca Alkaloid. Vinca alkaloids have the following general structure. They are indole-dihydroindole dimers.

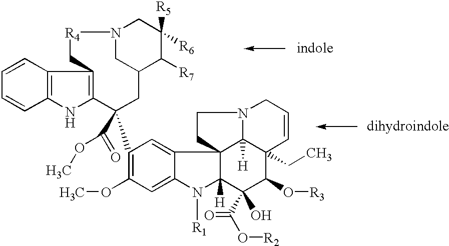

As disclosed in U.S. Pat. Nos. 4,841,045 and 5,030,620, R₁ can be a formyl or methyl group or alternately H. R₁ could also be an alkyl group or an aldehyde-substituted alkyl (e.g., CH₂CHO). R₂ is typically a CH₃ or NH₂ group. However it can be alternately substituted with a lower alkyl ester or the ester linking to the dihydroindole core may be substituted with C(O)—R where R is NH₂, an amino acid ester or a peptide ester. R₃ is typically C(O)CH₃, CH₃ or H. Alternately a protein fragment may be linked by a bifunctional group such as maleoyl amino acid. R₃ could also be substituted to form an alkyl ester which may be further substituted. R₄ may be —CH₂— or a single bond. R₅ and R₆ may be either H, OH or a lower alkyl, typically —CH₂CH₃. Alternatively R₆ and R₇ may together form an oxetane ring. R₇ may alternately be H. Further substitutions include molecules wherein methyl groups are substituted with other alkyl groups, and whereby unsaturated rings may be derivatized by the addition of a side group such as an alkane, alkene, alkyne, halogen, ester, amide or amino group.

Exemplary Vinca Alkaloid are vinblastine, vincristine, vincristine sulfate, vindesine, and vinorelbine, having the structures:

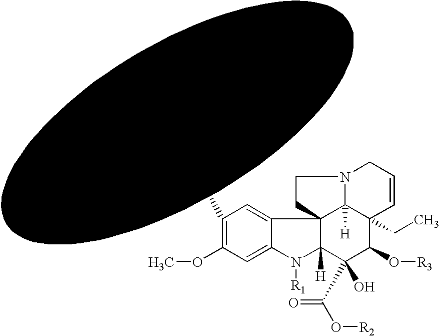

| | R₁ | R₂ | R₃ | R₄ | R₅ |
|---|---|---|---|---|---|
| Vinblastine: | CH₃ | CH₃ | C(O)CH₃ | OH | CH₂ |
| Vincristine: | CH₂O | CH₃ | C(O)CH₃ | OH | CH₂ |
| Vindesine: | CH₃ | NH₂ | H | OH | CH₂ |
| Vinorelbine: | CH₃ | CH₃ | CH₃ | H | single bond |

Analogs typically require the side group (shaded area) in order to have activity.

Vinca alkaloids act as anti-microtubule agents by inhibiting polymerization of microtubules.

C. Camptothecin

In another aspect, the anti-microtubule agent is Camptothecin, or an analog or derivative thereof. Camptothecins have the following general structure.

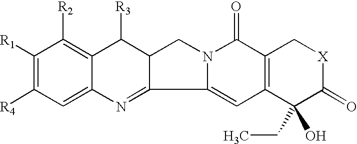

In this structure, X is typically 0, but can be other groups, e.g, NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g, NO₂, NH₂, halogen (as disclosed in, e.g, U.S. Pat. No. 5,552,156) or a short alkane containing these groups. R₃ is typically H or a short alkyl such as C₂H₅. R₄ is typically H but may be other groups, e.g., a methylenedioxy group with R₁ Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

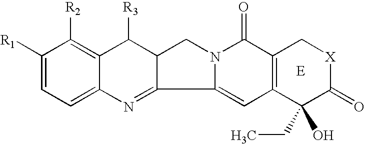

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity.

D. Nitrogen Mustards

In another aspect, the Anti-microtubule agent is a Nitrogen Mustard. Many suitable Nitrogen Mustards are known and are suitably used as a Anti-microtubule agent in the present invention. Suitable nitrogen mustards are also known as cyclophosphamides.

A preferred nitrogen mustard has the general structure:

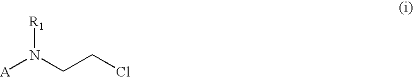

Where A is:

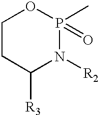

or —CH₃ or other alkane, or chloronated alkane, typically CH₂CH(CH₃)Cl, or a polycyclic group such as B, or a substituted phenyl such as C or a heterocyclic group such as D.

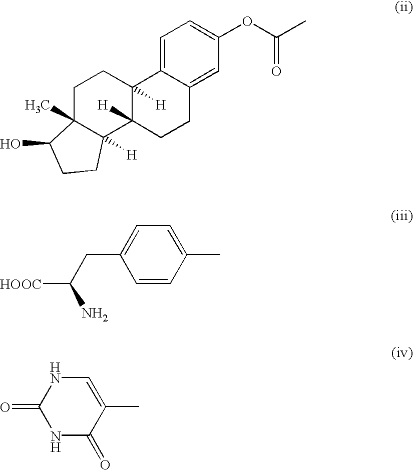

Suitable nitrogen mustards are disclosed in U.S. Pat. No. 3,808,297, wherein A is:

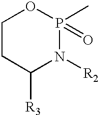

R₁₋₂ are H or CH₂CH₂Cl; R₃ is H or oxygen-containing groups such as hydroperoxy; and R₄ can be alkyl, aryl, heterocyclic.

The cyclic moiety need not be intact. See, e.g., U.S. Pat. Nos. 5,472,956, 4,908,356, 4,841,085 that describe the following type of structure:

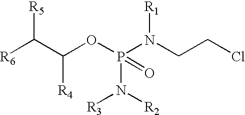

wherein R₁ is H or CH₂CH₂Cl, and R₂₋₆ are various substituent groups.

Exemplary nitrogen mustards include methylchloroethamine, and analogs or derivatives thereof, including methylchloroethamine oxide hydrohchloride, Novembichin, and Mannomustine (a halogenated sugar). Exemplary compounds have the structures:

| 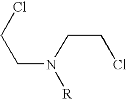 | | 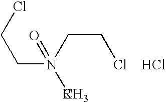 |
|---|---|---|
| | R | |
| Mechlorethanime | CH₃ | Mechlorethanime Oxide HCl |
| Novembichin | CH₂CH(CH₃)Cl | |

The Nitrogen Mustard may be Cyclophosphamide, Ifosfamide, Perfosfamide, or Torofosfamide, where these compounds have the structures:

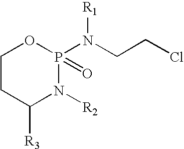

| | R₁ | R₂ | R₃ |
|---|---|---|---|
| Cyclophosphamide | H | CH₂CH₂Cl | H |
| Ifosfamide | CH₂CH₂Cl | H | H |
| Perfosfamide | CH₂CH₂Cl | H | OOH |
| Torofosfamide | CH₂CH₂Cl | CH₂CH₂Cl | H |

The Nitrogen Mustard may be Estramustine, or an analog or derivative thereof, including Phenesterine, Prednimustine, and Estramustine PO₄. Thus, suitable nitrogen mustard type Anti-microtubule agents of the present invention have the structures:

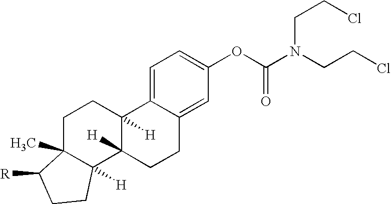

| | R |
|---|---|
| Estramustine | OH |
| Phenesterine | C(CH₃)(CH₂)₃CH(CH₃)₂ |

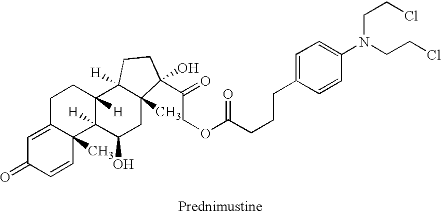

The Nitrogen Mustard may be Chlorambucil, or an analog or derivative thereof, including Melphalan and Chlormaphazine. Thus, suitable nitrogen mustard type Anti-microtubule agents of the present invention have the structures:

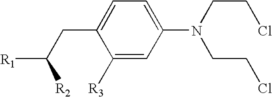

| | R₁ | R₂ | R₃ |
|---|---|---|---|
| Chlorambucil | CH₂COOH | H | H |
| Melphalan | COOH | NH₂ | H |

| | | |
|---|---|---|
| Chlornaphazine | H | together forms |
| | | a benzene ring |

The Nitrogen Mustard may be Uracil Mustard, which has the structure:

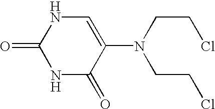

E. Podophyllotoxins

In another aspect, the anti-microtubule agent is a Podophyllotoxin, or a derivative or an analog thereof. Exemplary compounds of this type are Etoposide or Teniposide, which have the following structures:

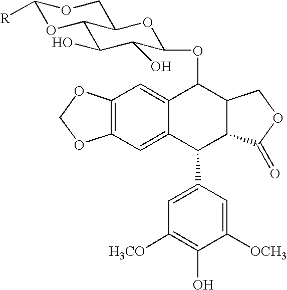

| | R |
|---|---|
| Etoposide | CH₃ |
| Teniposide | 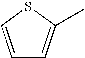 |

### (II) Anti-Microtubule Agent Compositions and Formulations

As noted above, therapeutic anti-microtubule agents described herein may be formulated in a variety of manners, and thus may additionally comprise a carrier. In this regard, a wide variety of carriers may be selected of either polymeric or non-polymeric origin. The polymers and non-polymer based carriers and formulations which are discussed in more detail below are provided merely by way of example, not by way of limitation.

Within one embodiment of the invention a wide variety of polymers may be utilized to contain and/or deliver one or more of the anti-microtubule agents discussed above, including for example both biodegradable and non-biodegradable compositions. Representative examples of biodegradable compositions include albumin, collagen, gelatin, chitosan, hyaluronic acid, starch, cellulose and derivatives thereof (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate), alginates, casein, dextrans, polysaccharides, fibrinogen, poly(L-lactide), poly(D,L lactide), poly(L-lactide-co-glycolide), poly(D,L-lactide-co-glycolide), poly(glycolide), poly(trimethylene carbonate), poly(hydroxyvalerate), poly(hydroxybutyrate), poly(caprolactone), poly(alkylcarbonate) and poly(orthoesters), polyesters, poly(hydroxyvaleric acid), polydioxanone, poly(malic acid), poly(tartronic acid), polyanhydrides, polyphosphazenes, poly(amino acids), copolymers of such polymers and blends of such polymers (see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar 59:173-196, 1990; Holland et al., J. Controlled Release 4:155-0180, 1986). Representative examples of nondegradable polymers include poly(ethylene-co-vinyl acetate) (“EVA”) copolymers, silicone rubber, acrylic polymers (e.g., polyacrylic acid, polymethylacrylic acid, poly(hydroxyethylmethacrylate), polymethylmethacrylate, polyalkylcyanoacrylate), polyethylene, polyproplene, polyamides (e.g., nylon 6,6), polyurethane (e.g., poly(ester urethanes), poly(ether urethanes), poly(ester-urea), poly(carbonate urethanes)), polyethers (e.g., poly(ethylene oxide), poly(propylene oxide), Pluronics and poly(tetramethylene glycol)) and vinyl polymers [e.g., polyvinylpyrrolidone, poly(vinyl alcohol), poly(vinyl acetate phthalate)]. Polymers may also be developed which are either anionic (e.g., alginate, carrageenin, carboxymethyl cellulose and poly(acrylic acid), or cationic (e.g., chitosan, poly-L-lysine, polyethylenimine, and poly (allyl amine)) (see generally, Dunn et al., J. Applied Polymer Sci. 50:353-365, 1993; Cascone et al., J. Materials Sci.: Materials in Medicine 5:770-774, 1994; Shiraishi et al., Biol. Pharm. Bull. 16(11):1164-1168, 1993; Thacharodi and Rao, Int'l J. Pharm. 120:115-118, 1995; Miyazaki et al., Int'l J. Pharm. 118:257-263, 1995). Particularly preferred polymeric carriers include poly(ethylene-co-vinyl acetate), polyurethane, poly(D,L-lactic acid) oligomers and polymers, poly(L-lactic acid) oligomers and polymers, poly(glycolic acid), copolymers of lactic acid and glycolic acid, poly(caprolactone), poly(valerolactone), polyanhydrides, copolymers of poly(caprolactone) or poly(lactic acid) with a polyethylene glycol (e.g., MePEG), and blends thereof.

Other representative polymers include carboxylic polymers, polyacetates, polyacrylamides, polycarbonates, polyethers, polyesters, polyethylenes, polyvinylbutyrals, polysilanes, polyureas, polyurethanes, polyoxides, polystyrenes, polysulfides, polysulfones, polysulfonides, polyvinylhalides, pyrrolidones, rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxy, melamine, other amino resins, phenolic polymers, and copolymers thereof, water-insoluble cellulose ester polymers (including cellulose acetate propionate, cellulose acetate, cellulose acetate butyrate, cellulose nitrate, cellulose acetate phthalate, and mixtures thereof), polyvinylpyrrolidone, polyethylene glycols, polyethylene oxide, polyvinyl alcohol, polyethers, polysaccharides, hydrophilic polyurethane, polyhydroxyacrylate, dextran, xanthan, hydroxypropyl cellulose, methyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, hydroxyethyl cellulose, cellulose nitrate, cellulose acetate, cellulose acetate butyrate, cellulose acetate propionate, polyurethane, polyacrylate, natural and synthetic elastomers, rubber, acetal, nylon, polyester, styrene polybutadiene, acrylic resin, polyvinylidene chloride, polycarbonate, homopolymers and copolymers of vinyl compounds, polyvinylchloride, polyvinylchloride acetate.

Representative examples of patents relating to polymers and their preparation include PCT Publication Nos. WO72827, 98/12243, 98/19713, 98/41154, 99/07417, 00/33764, 00/21842, 00/09190, 00/09088, 00/09087, 2001/17575 and 2001/15526 (as well as their corresponding U.S. applications), and U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,106,473, 6,110,483, 6,121,027, 6,156,345, and 6,214,901, which, as noted above, are all incorporated by reference in their entirety.

Polymers can be fashioned in a variety of forms, with desired release characteristics and/or with specific desired properties. For example, polymers can be fashioned to release a therapeutic agent upon exposure to a specific triggering event such as pH (see, e.g., Heller et al., “Chemically Self-Regulated Drug Delivery Systems,” in Polymers in Medicine III, Elsevier Science Publishers B. V., Amsterdam, 1988, pp. 175-188; Kang et al., J. Applied Polymer Sci. 48:343-354, 1993; Dong et al., J. Controlled Release 19:171-178, 1992; Dong and Hoffman, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH— and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid)-based polymers and derivatives (including, for example, homopolymers such as poly(aminocarboxylic acid), poly(acrylic acid), poly(methyl acrylic acid), copolymers of such homopolymers, and copolymers of poly(acrylic acid) and acrylmonomers such as those discussed above). Other pH sensitive polymers include polysaccharides such as carboxymethyl cellulose, hydroxypropylmethylcellulose phthalate, hydroxypropyl-methylcellulose acetate succinate, cellulose acetate trimellilate, chitosan and alginates. Yet other pH sensitive polymers include any mixture of a pH sensitive polymer and a water-soluble polymer.

Likewise, polymers can be fashioned which are temperature sensitive (see, e.g., Chen et al., “Novel Hydrogels of a Temperature-Sensitive Pluronic Grafted to a Bioadhesive Polyacrylic Acid Backbone for Vaginal Drug Delivery,” in Proceed. Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact. Mater 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oreg. Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, NY, pp. 822-823; Hoffman et al., “Characterizing Pore Sizes and Water ‘Structure’ in Stimuli-Responsive Hydrogels,” Center for Bioengineering, Univ. of Wash., Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102, 1994; Okano et al., J. Controlled Release 36:125-133, 1995; Chun and Kim, J. Controlled Release 38:39-47, 1996; D'Emanuele and Dinarvand, Intl J. Pharm. 118:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B.V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12): 1997-2002, 1995).

Representative examples of thermogelling polymers include homopolymers such as poly(N-methyl-N-n-propylacrylamide), poly(N-n-propylacrylamide), poly(N-methyl-N-isopropylacrylamide), poly(N-n-propylmethacrylamide), poly(N-isopropylacrylamide), poly(N, n-diethylacrylamide), poly(N-isopropylmethacrylamide), poly(N-cyclopropylacrylamide), poly(N-ethylmethyacrylamide), poly(N-methyl-N-ethylacrylamide), poly(N-cyclopropylmethacrylamide) and poly(N-ethylacrylamide). Moreover thermogelling polymers may be made by preparing copolymers between (among) monomers of the above, or by combining such homopolymers with other water-soluble polymers such as acrylmonomers (e.g., acrylic acid and derivatives thereof such as methylacrylic acid, acrylate and derivatives thereof such as butyl methacrylate, acrylamide, and N-n-butyl acrylamide).

Other representative examples of thermogelling cellulose ether derivatives such as hydroxypropyl cellulose, methyl cellulose, hydroxypropylmethyl cellulose, ethylhydroxyethyl cellulose, and Pluronics, such as F-127, L-122, L-92, L-81, and L-61.

A wide variety of forms may be fashioned by the polymers of the present invention, including for example, rod-shaped devices, pellets, slabs, particulates, micelles, films, molds, sutures, threads, gels, creams, ointments, sprays or capsules (see, e.g., Goodell et al., Am. J. Hosp. Pharm. 43:1454-1461, 1986; Langer et al., “Controlled release of macromolecules from polymers”, in Biomedical Polymers, Polymeric Materials and Pharmaceuticals for Biomedical Use, Goldberg, E. P., Nakagim, A. (eds.) Academic Press, pp. 113-137, 1980; Rhine et al., J. Pharm. Sci. 69:265-270, 1980;:Brown et al., J. Pharm. Sci. 72:1181-1185, 1983; and Bawa et al., J. Controlled Release 1:259-267, 1985). Anti-microtubule agents may be linked by occlusion in the matrices of the polymer, bound by covalent linkages, or encapsulated in microcapsules. Within certain preferred embodiments of the invention, therapeutic compositions are provided in non-capsular formulations, such as microspheres (ranging from nanometers to micrometers in size), pastes, threads or sutures of various size, films and sprays.

Other compositions which may be utilized to carrier and/or deliver the anti-microtubule agents provided herein include vitamin-based compositions (e.g., based on vitamins A, D, E and/or K, see, e.g., PCT publication Nos. WO 98/30205 and WO 00/71163) and liposomes (see, U.S. Pat. Nos. 5,534,499, 5,683,715, 5,776,485, 5,882,679, 6,143,321, 6,146,659, 6,200,598, and PCT Publication Nos. WO 98/34597, WO 99/65466, WO 00/01366, WO 00/53231, WO 99/35162, WO 00/117508, WO 00/125223, WO 00/149,268, WO 00/1565438, WO 00/158455,

Preferably, therapeutic compositions of the present invention are fashioned in a manner appropriate to the intended use. Within certain aspects of the present invention, the therapeutic composition should be biocompatible, and release one or more anti-microtubule agents over a period of several days to months. For example, “quick release” or “burst” therapeutic compositions are provided that release greater than 10%, 20% or 25% (w/v) of a therapeutic agent (e.g., paclitaxel) over a period of 7 to 10 days. Such “quick release” compositions should, within certain embodiments, be capable of releasing chemotherapeutic levels (where applicable) of a desired agent. Within other embodiments, “slow release” therapeutic compositions are provided that release less than 1% (w/v) of a therapeutic agent over a period of 7 to 10 days. Further, therapeutic compositions of the present invention should preferably be stable for several months and capable of being produced and maintained under sterile conditions.

Within certain aspects of the present invention, therapeutic compositions may be fashioned in any size ranging from 50 nm to 500 μm, depending upon the particular use. Alternatively, such compositions may also be readily applied as a “spray” which solidifies into a film or coating. Such sprays may be prepared from microspheres of a wide array of sizes, including for example, from 0.1 μm to 9 μm, from 10 μm to 30 μm and from 30 μm to 100 μm.

Therapeutic compositions of the present invention may also be prepared in a variety of “paste” or gel forms. For example, within one embodiment of the invention, therapeutic compositions are provided which are liquid at one temperature (e.g., temperature greater than 37° C.) and solid or semi-solid at another temperature (e.g., ambient body temperature, or any temperature lower than 37° C.). Also included are polymers, such as Pluronic F-127, which are liquid at a low temperature (e.g, 4° C.) and a gel at body temperature. Such “thermopastes” may be readily made given the disclosure provided herein.

Within yet other aspects of the invention, the therapeutic compositions of the present invention may be formed as a film. Preferably, such films are generally less than 5, 4, 3, 2 or 1 mm thick, more preferably less than 0.75 mm or 0.5 mm thick, and most preferably less than 500 μm. Such films are preferably flexible with a good tensile strength (e.g., greater than 50, preferably greater than 100, and more preferably greater than 150 or 200 N/cm²), good adhesive properties (i.e., readily adheres to moist or wet surfaces), and have controlled permeability.

Within further aspects of the invention, the therapeutic compositions may be formulated for topical application. Representative examples include: ethanol; mixtures of ethanol and glycols (e.g., ethylene glycol or propylene glycol); mixtures of ethanol and isopropyl myristate or ethanol, isopropyl myristate and water (e.g., 55:5:40); mixtures of ethanol and eineol or D-limonene (with or without water); glycols (e.g., ethylene glycol or propylene glycol) and mixtures of glycols such as propylene glycol and water, phosphatidyl glycerol, dioleoylphosphatidyl glycerol, Transcutol®, or terpinolene; mixtures of isopropyl myristate and 1-hexyl-2-pyrrolidone, N-dodecyl-2-piperidinone or 1-hexyl-2-pyrrolidone. Other excipients may also be added to the above, including for example, acids such as oleic acid and linoleic acid, and surfactants, such as sodium lauryl sulfate. For a more detailed description of the above, see generally, Hoelgaard et al., J. Contr. Rel. 2:111, 1985; Liu et al., Pharm. Res. 8:938, 1991; Roy et al, J. Pharm. Sci. 83:126, 1991; Ogiso et al., J. Pharm. Sci. 84:482, 1995; Sasaki et al., J. Pharm. Sci. 80:533, 1991; Okabe et al., J. Contr. Rel. 32:243, 1994; Yokomizo et al., J. Contr. Rel. 38:267, 1996; Yokomizo et al., J. Contr. Rel. 42:37, 1996; Mond et al., J. Contr Rel. 33:72, 1994; Michniak et al., J. Contr. Rel. 32:147, 1994; Sasaki et al., J. Pharm. Sci. 80:533, 1991; Baker & Hadgraft, Pharm. Res. 12:993, 1995; Jasti et al., AAPS Proceedings, 1996; Lee et al., AAPS Proceedings, 1996; Ritschel et al., Skin Pharmacol. 4:235, 1991; and McDaid & Deasy, Int. J. Pharm. 133:71, 1996.

Within certain embodiments of the invention, the therapeutic compositions can also comprise additional ingredients such as surfactants (e.g., Pluronics such as F-127, L-122, L-92, L-81, and L-61).

Within further aspects of the present invention, polymers are provided which are adapted to contain and release a hydrophobic compound, the carrier containing the hydrophobic compound in combination with a carbohydrate, protein or polypeptide. Within certain embodiments, the polymeric carrier contains or comprises regions, pockets or granules of one or more hydrophobic compounds. For example, within one embodiment of the invention, hydrophobic compounds may be incorporated within a matrix which contains the hydrophobic compound, followed by incorporation of the matrix within the polymeric carrier. A variety of matrices can be utilized in this regard, including for example, carbohydrates and polysaccharides, such as starch, cellulose, dextran, methylcellulose, and hyaluronic acid, proteins or polypeptides such as albumin, collagen and gelatin. Within alternative embodiments, hydrophobic compounds may be contained within a hydrophobic core, and this core contained within a hydrophilic shell.

Other carriers that may likewise be utilized to contain and deliver the anti-microtubule agents described herein include: hydroxypropyl β-cyclodextrin (Cserhati and Hollo, Int. J. Pharm. 108:69-75, 1994), liposomes (see, e.g., Sharma et al., Cancer Res. 53:5877-5881, 1993; Sharma and Straubinger, Pharm. Res. 11(60):889-896, 1994; WO 93/18751; U.S. Pat. No. 5,242,073), liposome/gel (WO 94/26254), nanocapsules (Bartoli et al., J. Microencapsulation 7(2):191-197, 1990), micelles (Alkan-Onyuksel et al., Pharm. Res. 11(2):206-212, 1994), implants (Jampel et al., Invest. Ophthalm. Vis. Science 34(11): 3076-3083, 1993; Walter et al., Cancer Res. 54:22017-2212, 1994), nanoparticles (Violante and Lanzafame PAACR), nanoparticles—modified (U.S. Pat. No. 5,145,684), nanoparticles (surface modified) (U.S. Pat. No. 5,399,363), taxol emulsion/solution (U.S. Pat. No. 5,407,683), micelle (surfactant) (U.S. Pat. No. 5,403,858), synthetic phospholipid compounds (U.S. Pat. No. 4,534,899), gas borne dispersion (U.S. Pat. No. 5,301,664), foam, spray, gel, lotion, cream, ointment, dispersed vesicles, particles or droplets solid- or liquid-aerosols, microemulsions (U.S. Pat. No. 5,330,756), polymeric shell (nano- and micro-capsule) (U.S. Pat. No. 5,439,686), taxoid-based compositions in a surface-active agent (U.S. Pat. No. 5,438,072), liquid emulsions (Tarr et al., Pharm Res. 4:62-165, 1987), nanospheres (Hagan et al., Proc. Intern. Symp. Control Rel. Bioact. Mater. 22, 1995; Kwon et al., Pharm Res. 12(2):192-195; Kwon et al., Pharm Res. 10(7):970-974; Yokoyama et al., J. Contr. Rel. 32:269-277, 1994; Gref et al., Science 263:1600-1603, 1994; Bazile et al., J. Pharm. Sci. 84:493-498, 1994) and implants (U.S. Pat. No. 4,882,168).

In another embodiment, intraoccular lens can also be loaded directly with an anti-microtubule agent. In this case, different organic solvents in which lenses are not soluble (e.g., methanol, ethanol) can be used to dissolved the agent. Solutions ranging from 0.1% to 30% can be used to incorporate the anti-microtubule agent into the lens. Intraoccular lenses suitable for loading with agents include hydrogel, polymethylmethacrylate and silicone. In another embodiment, an antimicrotubule agent-releasing polymeric delivery system previously described is attached, coated, sprayed, dipped on all or parts of the intraoccular lens or on the loops of the lens. After implantation of the lens in the eye of the patient (e.g., after cataract surgery) the agent is released from the lens at the appropriate release rate.

The anti-microtubule agents provided herein can also be formulated as a sterile composition (e.g., by treating the composition with ethylene oxide or by irradiation), packaged with preservatives or other suitable excipients suitable for administration to humans. Similarly, the devices provided herein (e.g., coated intraoccular lenses) may be sterilized and prepared suitable for implantation into humans.

### (III) Clinical Applications

In order to further the understanding of the invention, discussed in more detail below are various clinical applications for the compositions, methods and devices provided herein.

Briefly, as noted above, within one aspect of the invention methods are provided for treating or preventing uveitis comprising the step of administering to the patient an anti-microtubule agent. Within one embodiment, a formulation (e.g., micellar paclitaxel) can be given systemically by intravenous injection, by intramuscular injections or by oral, nasal, transdermal, inhalation or parenteral administration.

Within other embodiments, a formulation containing an antimicrotubule agent can be administered by local administration. Representative examples of local administration include eye drops, periocular injection or implantation and intraoccular injection or implantation.

Anti-microtubule agents can be given to treat uveitis after diagnosis, or, prophylatically before uveitis has occurred. Administration can be by means of a coated intraoccular lens implanted at the time of cataract surgery.

It should be readily evident to one of skill in the art that any of the previously mentioned anti-microtubule agents, or derivatives and analogues thereof, can be utilized to create variation of the above compositions without deviating from the spirit and scope of the invention.

### EXAMPLES

### Example 1

### Manufacture of Micellar Paclitaxel

Poly(DL-lactide)-block-methoxypolyethylene glycol (PDLLA-block-MePEG) with a MePEG molecular weight of 2000 and a PDLLA:MePEG weight ratio 40:60 is used as the micellar carrier for the solubilization of paclitaxel. PDLLA-MePEG 2000-40/60 (polymer) is an amphiphilic diblock copolymer that dissolves in aqueous solutions to form micelles with a hydrophobic PDLLA core and hydrophilic MePEG shell. Paclitaxel is physically trapped in the hydrophobic PDLLA core to achieve the solubilization.

The polymer was synthesized from the monomers methoxypolyethylene glycol and DL-lactide in the presence of 0.5% w/w stannous octoate through a ring opening polymerization. Stannous octoate acted as a catalyst and participated in the initiation of the polymerization reaction. Stannous octoate forms a number of catalytically reactive species which complex with the hydroxyl group of MePEG and provide an initiation site for the polymerization. The complex attacks the DL-lactide rings and the rings open up and are added to the chain, one-by-one, forming the polymer. The calculated molecular weight of the polymer is 3,333.

All reaction glassware was washed and rinsed with Sterile Water for Irrigation, USP, dried at 37° C., followed by depyrogenation at 250° C. for at least 1 hour. MePEG (240 g) and DL-lactide (160 g) were weighed and transferred to a round bottom glass flask using a stainless steel funnel. A 2 inch Teflon® coated magnetic stir bar was added to the flask. The flask was sealed with a glass stopper and then immersed to the neck in a 140° C. oil bath. After the MePEG and DL-lactide melted, 2 mL of 95% stannous octoate (catalyst) was added to the flask. The flask was vigorously shaken immediately after the addition to ensure rapid mixing and then returned to the oil bath. The reaction was allowed to proceed for an additional 6 hours with heat and stirring. The liquid polymer was then poured into a stainless steel tray, covered and left in a chemical fume hood overnight (about 16 hours). The polymer solidified in the tray. The top of the tray was sealed using Parafilm®. The sealed tray containing the polymer was placed in a freezer at −20±5° C. for at least 0.5 hour. The polymer was then removed from the freezer, broken up into pieces and transferred to glass storage bottles and stored refrigerated at 2 to 8° C.

Preparation of a 50 mg/m2 Dose

Preparation of the bulk and filling of paclitaxel/polymer matrix was accomplished essentially as follows. Reaction glassware was washed and rinsed with Sterile Water for Irrigation USP, and dried at 37° C., followed by depyrogenation at 250° C. for at least 1 hour. First, a phosphate buffer (0.08 M, pH 7.6) was prepared. The buffer was dispensed at the volume of 10 mL per vial. The vials were heated for 2 hours at 90° C. to dry the buffer. The temperature was then raised to 160° C. and the vials dried for an additional 3 hours.

The polymer was dissolved in acetonitrile at 15% w/v concentration with stirring and heat. The polymer solution was then centrifuged at 3000 rpm for 30 minutes. The supernatant was poured off and set aside. Additional acetonitrile was added to the precipitate and. centrifuged a second time at 3000 rpm for 30 minutes. The second supernatant was pooled with the first supernatant. Paclitaxel was weighed and then added to the supernatant pool. The solution was brought to the final desired volume with acetonitrile.

The paclitaxel/polymer matrix solution is dispensed into the vials containing previously dried phosphate buffer at a volume of 10 ML per vial. The vials are then vacuum dried to remove the acetonitrile. The paclitaxel/polymer matrix is then terminally sterilized by irradiation with at. least 2.5 Mrad Cobalt-60 (Co-60) x-rays.

### Example 2

### Prevention of Experimental Autoimmmune Uveitis by Intraperitoneal Micellar Paclitaxel

Twenty-five male Lewis rats weighing 200 g-250 g are immunized by injecting into each footpad 0.1 mL of an emulsion containing 15 μg of S-antigen in phosphate-buffered saline (PBS) mixed with an equal volume of complete Freund's adjuvant augmented with H37Ra Mycobacterium tuberculosis to a concentration of 2.5 mg/mL (Paletine et al., 1987 JCI, 1078-1081). Rats are then divided into 5 groups of 5 animals. Micellar (Cremophor-free) paclitaxel was administered intraperitoneally (i.p.), every four days at 5 mg/kg (group 1), 10 mg/kg (group 2) and 15 mg/kg (group 3) starting on the day of immunization. Animals in group 4 are injected i.p. with vehicle micelles devoid of paclitaxel. Animals in group 5 are injected i.p. with PBS. At 16 days after immunization the animals are sacrificed with CO₂. The eyes are removed and fixed in 4% glutaraldehyde, embedded in glycol methacrylate, sectioned and stained with hematoxylin and eosin. The presence of ocular inflammation as defined by the presence of intraoccular inflammatory cells and photoreceptors destruction is graded by an observer blinded to the treatment groups.

### Example 3

### Prevention of Experimental Autoimmune Uveitis With Intravenous Micellar Paclitaxel

Autoimmune uveitis is induced in 25 rats under anesthesia by injecting into each footpad 0.1 ml of an emulsion containing 15 μg of S-antigen in phosphate-buffered saline (PBS) mixed with an equal volume of complete Freund's adjuvant augmented with H37Ra Mycobacterium tuberculosis to a concentration of 2.5 mg/mL (Paletine et al., 1987 JCI, 1078-1081).

Micellar paclitaxel is constituted with 2.1 mL of 0.9% Sodium Chloride Injection, USP with heating in a water bath, to a final paclitaxel concentration of 5 mg/mL. Sufficient formulation is drawn into a 1 mL syringe with a 26 gauge needle to deliver a volume adjusted to 0.6 mL to 0.7 mL with 0.9% Sodium Chloride Injection, USP. The entire dose is administered as a slow infusion over approximately 1 minute every other day for 16 days starting on the day of immunization. Rats are divided into five groups of five animals consisting of a saline-injected group, a control micelle group and three micellar paclitaxel groups (1 mg/kg, 5 mg/kg and 10 mg/kg). At the time of sacrifice, the animals are euthanized with CO₂. The eyes are removed and fixed in 4% glutaraldehyde, embedded in glycol methacrylate, sectioned and stained with hematoxylin and eosin. The presence of ocular inflammation as defined by the presence of intraoccular inflammatory cells and photoreceptor destruction is graded by an observer blinded to the treatment groups.

### Example 4

### Preparation of Paclitaxel-Loaded Polycaprolactone Implants

Five grams of polycaprolactone (mol. wt. 10,000 to 20,000; Polysciences, Warrington Penn. USA) are weighed into four 20-mL glass scintillation vials that are placed into a 600-mL beakers containing 50 mL of water. The beakers are gently heated to 65° C. and held at that temperature for 20 minutes until the polymer has melted. A known weight of paclitaxel is thoroughly mixed into 3 of the vials at 65° C. to obtain loadings (w/w) of 0.1%, 1% and 10%. No paclitaxel is added to the fourth vial (control polymer). The melted polymer solutions are poured into pre-warmed (60° C. oven) 2×2×2 mm molds containing a 6-0 Dacron suture. The suture is embedded in the polymer that is allowed to cool until solidified. Implants are sterilized with ethylene oxide and kept at 4° C. until surgery.

### Example 5

### Manufacture of Paclitaxel-Loaded Ethylene Vinyl Acetate Implants

A total mass of ethylene vinyl acetate (EVA) and paclitaxel of 250 mg is dissolved in 5 mL dichloromethane (DCM). Amounts of paclitaxel of 0.25 mg, 2.5 mg and 25 mg are used in these solutions to yield paclitaxel loadings of 0.1%, 1% and 10%. A control (devoid of paclitaxel) 5% solution of EVA in DCM (w/v) is also prepared. Aliquots of each solution are slowly pipetted into 2×2×2 mm molds and allowed to evaporate overnight. The EVA implants are then sterilized with ethylene oxide and kept at 4° C. until surgery.

### Example 6

### Prevention of Endotoxin-Induced Uveitis by Intravitreal Sustained-Release Paclitaxel Implants

Seventy-five female New Zealand White rabbits weighing 2.5 to 3 kg are injected subcutaneously with 10 mg of Mycobacterium tuberculosis H37Ra antigen suspended in 0.5-mL mineral oil. Fourteen days after tuberculin antigen injection the animals are anesthetized. A 5-mm peritomy is made at the superotemporal quadrant of the right eye. A 3-mm sclerotomy is created 1 to 2 mm from the limbus. A sustained-release paclitaxel device (EVA alone, 0.1% paclitaxel in EVA, 1% paclitaxel in EVA or 10% paclitaxel in EVA) is inserted into the vitreous cavity through the sclerotomy and is suspended at the sclerotomy site by a 6-0 Dacron suture (n=15 in each group). An additional group of sham treated animals receive no treatment (n=15). The sclerotomy and peritomy are then closed with 7-0 Vicryl sutures. One drop of topical gentamycin solution is instilled into the eyes after surgery for infection prophylaxis.

A microparticulate suspension of M. tuberculosis H37Ra antigen is prepared by ultrasonicating a suspension of the crude extract in sterile balance salt solution. Seven days after device implantation, 50 μg of antigen suspended in 0.1 mL of balanced salt solution is injected into the vitreous cavity of the right eye of all rabbit.

All rabbits are examined with slit lamp biomicroscopy and indirect ophthalmoscopy by a masked observer 3, 7 and 14 days after the intravitreal challenge. Comeal neovascularization, iris congestion, anterior chamber flares and vitreous opacity are graded following standard scales (Jaffe et al, 1998 Ophthalmology 105:46-56).

Five rabbits in each group are chosen randomly 3, 7 and 14 days after the intravitreal challenge for aqueous protein measurement and cell count. Animals are anesthetized and aqueous humor is aspirated from the right eye of each rabbit with a heparin-rinsed syringe connected to a 27-gauge needle. Aqueous cell count is measured by hemocytometry. One drop of aqueous is placed on a microscope slide and stained with Wright stain for differential cell count. The remaining aqueous is centrifuged. The supernatant is used for measurement of protein content in the aqueous using a kit (Bio-Rad, Richmond, Calif.) with bovine serum albumin as a standard dilution reference curve. After removal of the aqueous humor, animals are sacrificed by intravenous sodium pentobarbital injection and the right eye is enucleated for histology examination. Eyes are fixed in 10% formaldehyde and embedded in paraffin. Sections are cut through the entire globe orientated along the optic nerve and medullary ray.

### Example 7

### Therapeutic Agent Encapsulation in Polycaprolactone Micro Spheres

Reagents used in these experiments include: polycaprolactone (PCL; molecular weight 35,000-45,000) purchased from Polysciences (Warrington, Pa.), dichloromethane (DCM) from Fisher Scientific Co., Canada; polyvinyl alcohol (PVP) (molecular weight 12,000-18,000, 99% hydrolysed) from Aldrich Chemical Co. (Milwaukee, Wis.), and paclitaxel from Sigma Chemical Co. (St. Louis, Mo.). Unless otherwise stated all chemicals and reagents are used as supplied. Distilled water is used throughout.

A. Microsphere Preparation

5% w/w paclitaxel-loaded microspheres were prepared by dissolving 10 mg of paclitaxel and 190 mg of PCL in 2 mL of DCM, adding to 100 mL of 1% PVP aqueous solution and stirring at 1000 rpm at 25° C. for 2 hours. The suspension of microspheres was centrifuged at 1000×g for 10 minutes (Beckman GPR), the supernatant removed and the microspheres washed three times with water. The washed microspheres were air-dried overnight and stored at room temperature. Control microspheres (paclitaxel absent) were prepared as described above. Microspheres containing 1% and 2% paclitaxel were also prepared. Microspheres were sized using an optical microscope with a stage micrometer.

B. Encapsulation efficiency

A known weight of drug-loaded microspheres (about 5 mg) was dissolved in 8 mL of acetonitrile and 2 mL distilled water was added to precipitate the polymer. The mixture was centrifuged at 1000 g for 10 minutes and the amount of paclitaxel encapsulated was calculated from the absorbance of the supernatant measured in a UV spectrophotometer (Hewlett-Packard 8452A Diode Array Spectrophotometer) at 232 nm.

C. Drug Release Studies

About 10 mg of paclitaxel-loaded microspheres were suspended in 20 mL of 10 mM PBS (pH 7.4) in screw-capped tubes. The tubes were tumbled end-over-end at 37° C. and at given time intervals 19.5 ml of supernatant was removed (after allowing the microspheres to settle at the bottom), filtered through a 0.45 μm membrane filter and retained for paclitaxel analysis. An equal volume of PBS was replaced in each tube to maintain sink conditions throughout the study. The filtrates were extracted with 3×1 mL DCM, the DCM extracts evaporated to dryness under a stream of nitrogen, redissolved in 1 mL acetonitrile and analyzed by HPLC using a mobile phase of water:methanol:acetonitrile (37:5:58) at a flow rate of 1 mL/minute (Beckman Isocratic Pump), a C8 reverse phase column (Beckman), and UV detection (Shimadzu SPD A) at 232 nm.

D. Scanning electron microscopy

Microspheres were placed on sample holders, sputter-coated with gold and then placed in a Philips 501B SEM operating at 15 kV.

E. Results

Microsphere size ranged from 30 to 100 μm, although there was evidence in all paclitaxel-loaded or control batches of some microspheres falling outside this range. The loading efficiency of PCL microspheres with paclitaxel was always greater than 95% for all drug loadings studied. Scanning electron microscopy demonstrated that the microspheres were all spherical and many showed a rough or pitted surface morphology. There was no evidence of solid drug on the surface of the microspheres.

The time courses of paclitaxel release from 1%, 2% and 5% loaded PCL microspheres were biphasic. There was an initial rapid release of paclitaxel or “burst phase” at all drug loadings. The burst phase occurred over 1-2 days at 1% and 2% paclitaxel loading and over 3-4 days for 5% loaded microspheres. The initial phase of rapid release was followed by a phase of significantly slower drug release. For microspheres containing 1% or 2% paclitaxel there was no further drug release after 21 days. At 5% paclitaxel loading, the microspheres had released about 20% of the total drug content after 21 days.

F. Discussion

The solvent evaporation method of manufacturing paclitaxel-loaded microspheres produced very high paclitaxel encapsulation efficiencies ranging from 95 to 100%. This was due to the hydrophobic nature of paclitaxel that favored partitioning in the organic solvent phase containing the polymer.

The biphasic release profile for paclitaxel was typical of the release pattern for many drugs from biodegradable polymer matrices. Polycaprolactone is an aliphatic polyester which can be degraded by hydrolysis under physiological conditions and it is non-toxic and tissue compatible. The degradation of PCL is significantly slower than that of the extensively investigated polymers and copolymers of lactic and glycolic acids and is therefore suitable for the design of long-term drug delivery systems. The initial rapid or burst phase of paclitaxel release was thought to be due to diffusional release of the drug from the superficial region of the microspheres (close to the microsphere surface). Release of paclitaxel in the second (slower) phase of the release profiles was not likely due to degradation or erosion of PCL because studies have shown that under in vitro conditions in water there was no significant weight loss or surface erosion of PCL over a 7.5-week period. The slower phase of paclitaxel release was probably due to dissolution of the drug within fluid-filled pores in the polymer matrix and diffusion through the pores. The greater release rate at higher paclitaxel loading was probably a result of a more extensive pore network within the polymer matrix.

### Example 8

### Manufacture of Paclitaxel-Loaded Lactic Acid-Glycolic Acid Copolymers (PLGA) Microspheres

A. Method

Microspheres were manufactured in the size ranges 0.5 to 10 μm, 10-20 μm and 30-100 μm using standard methods (polymer was dissolved in dichloromethane and emulsified in a polyvinyl alcohol solution with stirring as previously described in PCL or PDLLA microspheres manufacture methods). Various ratios of PLLA to GA were used as the polymers with different molecular weights (given as Intrinsic Viscosity (I.V.))

B. Result

Microspheres were manufactured successfully from the following starting polymers:

| PLLA | GA | I.V. |
|---|---|---|
| 50 | 50 | 0.74 |
| 50 | 50 | 0.78 |
| 50 | 50 | 1.06 |
| 65 | 35 | 0.55 |
| 75 | 25 | 0.55 |
| 85 | 15 | 0.56 |

Paclitaxel at 10% or 20% loadings was successfully incorporated into all these microspheres. Microspheres were then sterilized with ethylene oxide and kept at 4° C. until surgery.

### Example 9

### Manufacture of Paclitaxel-Loaded Polyethyleneglycol (PEG) Microspheres

Microspheres containing 10 or 20% paclitaxel in PEG (M.W.=20,000) were prepared by the solvent evaporation method. Briefly, the appropriate amounts of paclitaxel and 0.5 g PEG were dissolved in 3 ml of acetone. This solution was emulsified into 100 mL of light mineral oil containing 0.5 g of Span 80. The mixture was stirred until microspheres formed (about 1.5 hours). The mixture was centrifuged at 2,000 rpm for 5 minutes and the oil decanted. The microspheres were washed with petroleum ether and then with ethanol and subsequently dried. The yield of microspheres was 94% and the encapsulation efficiency was 64%. Microspheres were then sterilized with ethylene oxide and kept at 4° C. until surgery.

### Example 10

### Manufacture of Paclitaxel-Loaded Chitosan Microspheres

Fifty milliliters of paraffin oil (Fisher Scientific) was placed in a 100 mL beaker at 60° C. and 0.5 mL of Span 80 (Fisher Scientific) was added. The mixture was stirred at 700 rpm. In a separate vial, chitosan (Fluka, low molecular weight) was dissolved in a 2% acetic acid (Fisher Scientific) at 25 mg/mL by stirring for 2 hours. This solution was diluted to 12.5 mg/mL with water. 6.25 mg of paclitaxel was then added into 5 mL of the 12.5 mg/mL chitosan solution (10% w/w paclitaxel to chitosan) together with 25 μL of Tween 40 (Fisher Scientific) and the suspension was homogenized using a polytron set at “mark 2” for 30 seconds. The chitosan-paclitaxel suspension was poured slowly into the paraffin and stirred for 5 hours. The microspheres were then washed three times in hexane and air-dried. Microspheres were then sterilized with ethylene oxide and kept at 4° C. until surgery.

The encapsulation efficiency of paclitaxel in the chitosan microspheres was determined by dissolution of 10 mg microspheres in 10 mL of 2% acetic acid followed by extraction and phase separation of paclitaxel in 1 ml of dichloromethane.

The release rate of paclitaxel in the chitosan microspheres was measured by adding 10 mg of the microspheres to a 15 mL Teflon® capped tube followed by 10 mL of phosphate buffer saline (pH=7.4). The tube was tumbled at 8 rpm at 37° C. for specified times. The tube was then centrifuged at 1000×g and the supernatant was collected for analysis of released drug. 10 mL of fresh phosphate buffer saline was added back to the tubes to retain sink condition in the release study.

### Example 11

### Manufacture of Paclitaxel-Loaded Hyaluronic Acid Microspheres

Two hundred milligrams of hyaluronic acid (sodium salt) was dissolved in 10 mL of distilled water overnight. 3.3 mg of paclitaxel (Hauser Chemical Company, Boulder Colo.) was placed in a 2 mL homogenizer and 1 mL of water was added. The paclitaxel was hand homogenized for 2 minutes to reduce the particle size. Immediately before the experiment, the homogenized paclitaxel was added to 3.3 mL of hyaluronic acid solution and mixed together using a spatula. 50 mL of light paraffin oil (Fisher Scientific) containing 250 μL of span 80 (Fisher Scientific) was stirred at 600 rpm at 50° C. using a propeller type overhead stirrer (Fisher Scientific) in a 100 mL beaker on a heating block. The hyaluronic acid-paclitaxel solution was added to the paraffin and allowed to stir for 5 hours at 50° C. The contents were allowed to settle under gravity and then washed three times with hexane. The resulted hyaluronic acid-paclitaxel microspheres (10 to 100 μm) contained 0.7% paclitaxel by weight. Microspheres were then sterilized with ethylene oxide and kept at 4° C. until surgery.

### Example 12

### Prevention of Endotoxin-Induced Uveitis With Intravitreal Paclitaxel Injection

Sixty female New Zealand White rabbits weighing 2.5 to 3 kg are anesthetized. Amethocaine (0.5%) is administered topically to the eyes to supplement general anesthesia. The right eye is exposed by retracting the upper lid. A 30-gauge needle is inserted transconjunctivally at the 12 o'clock position, 3 to 4 mm posterior to the limbus. Twenty μL liter of endotoxin (100 ng lipopolysaccharide from Salmonella typhimurium) and experimental solution (lug, 10 μg or 100 μg paclitaxel in micelles or hyaluronic acid microspheres) are injected using a disposable needle. The left eye is injected with 20 μL of endotoxin and control vehicle (micelles or hyaluronic acid microspheres devoid of paclitaxel). The animals are recovered from anesthesia. In each treatment group, 5 animals were sacrificed at 24 hours and 48 hours after injection. Immediately after death, 200 to 250 μL of aqueous humour was aspirated from the anterior chamber using a 30-gauge disposable insulin syringe. Aqueous humour cell count is performed using a hemoctometer. Differential cell count is performed after staining with Giemsa. The eyes are enucleated following aqueous humour sampling and are stored in 10% buffered formalin for 24 hours. Hematoxylin and eosin-stained sections cut at 5 μm thickness are prepared from paraffin-embedded blocks of the enucleated eyes. Sections are examined for the presence of keratic precipitates, inflammatory cells and altered vascularity and are graded using standard scales (Verma et al 1999 IOVS 40(11):2465-2470).

### Example 13

### Prevention of Chronic Endotoxin-Induced Uveitis by Intravitreal Paclitaxel Injection

Ninety female New Zealand White rabbits weighing 2.5 to 3 kg are injected subcutaneously with 10 mg of Mycobacterium tuberculosis H37Ra antigen suspended in 0.5-mL mineral oil.

A microparticulate suspension of M. tuberculosis H37Ra antigen is prepared by ultrasonicating a suspension of the crude extract in sterile balance salt solution. Fourteen days after tuberculin antigen injection the animals are anesthetized and divided into 6 treatment groups of 15 animals (1 μg, 10 μg or 100 μg paclitaxel in 2% paclitaxel PCL microspheres or in 10% paclitaxel PLGA microspheres). In each animal, microspheres and 50 μg of antigen suspended in 0.1 mL of balanced salt solution are injected into the vitreous cavity of the right eye. The left eye is injected with the corresponding control microspheres (devoid of paclitaxel) and 50 μg of antigen in 0.1 mL of balanced salt solution.

All rabbits are examined with slit lamp biomicroscopy and indirect ophthalmoscopy by a masked observer 3, 7 and 14 days after the intravitreal challenge. Corneal neovascularization, iris congestion, anterior chamber flares and vitreous opacity are graded following standard scales (Jaffe et al 1998 Ophthalmology 105:46-56).

Five rabbits in each group are chosen randomly 3, 7 and 14 days after the intravitreal challenge for aqueous protein measurement and cell count. Animals are anesthetized and aqueous humor is aspirated from the right eye of each rabbit with a heparin-rinsed syringe connected to a 27-gauge needle. Aqueous cell count is measured by hemocytometry. One drop of aqueous is placed on a microscope slide and stained with Wright stain for differential cell count. The remaining aqueous is centrifuged. The supernatant is used for measurement of protein content in the aqueous using a kit (Bio-Rad, Richmond, Calif.) with bovine serum albumin as a standard dilution reference curve. After removal of the aqueous humor, animals are sacrificed by intravenous sodium pentobarbital injection and the right eye is enucleated for histology examination. Eyes are fixed in 10% formaldehyde and embedded in paraffin. Sections are cut through the entire globe orientated along the optic nerve and medullary ray. Sections are examined for the presence of keratic precipitates, inflammatory cells and altered vascularity and are graded using standard scales (Verma et al., IOVS 40(11):2465-2470, 1999).

### Example 14

### Prevention of Uveitis With Paclitaxel-Loaded Intraocular Lenses

Solutions of 0.1%, 1% and 10% paclitaxel are prepared in methanol. Intraocular lenses are soaked for 24 hours in the different solutions. The lenses are then removed and dried in a vacuum oven for 24 hours. They are then sterilized in ethylene oxide and kept at 4C until surgery.

Forty-five female New Zealand White rabbits weighing 2.5 to 3 kg are anesthetized. After a small upper cuvilinear buttonhole anterior capsulotomy is made, endocapsular phacoemulsification is performed. Posterior chamber IOLs coated with 0.1% (n=15), 1% (n=15) and 10% paclitaxel (n=15) are implanted in the capsular bag of the right eye. Control uncoated lenses are implanted following the same procedure in the left eye.

Five rabbits in each group are chosen randomly 3, 7 and 14 days after surgery for aqueous protein measurement and cell count. Animals are anesthetized and aqueous humor is aspirated from both eyes in each rabbit with a heparin-rinsed syringe connected to a 27-gauge needle. Aqueous cell count is measured by hemocytometry. One drop of aqueous is placed on a microscope slide and stained with Wright stain for differential cell count. The remaining aqueous is centrifuged. The supernatant is used for measurement of protein content in the aqueous using a kit (Bio-Rad, Richmond, Calif.) with bovine serum albumin as a standard dilution reference curve. After removal of the aqueous humor, animals are sacrificed by intravenous sodium pentobarbital injection and both eyes are enucleated for histology examination. Eyes are fixed in 10% formaldehyde and embedded in paraffin. Sections are cut through the entire globe orientated along the optic nerve and medullary ray. Sections are examined for the presence of keratic precipitates, inflammatory cells and altered vascularity and are graded using standard scales (Verma et al., IOVS 40(11): 2465-2470, 1999).

### Example 15

### Method to Attach an Antimicrotubule Delivery System to an Intraocular Lens

Weigh a known mass of polylactic acid directly into a 20-mL glass scintillation vial and add sufficient dichloromethane (DCM) to achieve a 10% w/v solution. Cap the vial and mix the solution. Add sufficient paclitaxel to the solution to achieve the desired final paclitaxel concentration. Hand-shake or vortex to dissolve paclitaxel in the solution. Let the solution sit for one hour (to diminish the presence of air bubbles) and then pour it slowly into a ring-shaped mold (8 mm outside diameter, 1×1 mm thick). Place the mold in the fume hood overnight. This will allow the DCM to evaporate. Peel the ring-shaped film out and glue it to the periphery of an intraoccular lens with fibrin adhesive. Sterilize the lens in ethylene oxide and store at 4° C. until surgery.

### Example 16

### Method to Coat Loops of Intraocular Lenses With an Antimicrotubule Delivery System

Weigh 2 g of EVA into a 20-mL glass scintillation vial and add 20 mL of dichloromethane. Cap the vial and leave it for 2 hours to dissolve (hand shake the vial frequently to assist the dissolving process). Weigh a known mass of paclitaxel directly into a 1-mL glass test tube and add 0.5 mL of the polymer solution. Using a glass Pasteur pipette, dissolve paclitaxel by gently pumping the polymer solution. Once paclitaxel is dissolved, hold the test tube in a near horizontal position (the sticky polymer solution will not flow out). Grab one loop of the lens with tweezers and insert the other loop into the tube. Allow the polymer solution to flow almost to the mouth of the test tube by angling the mouth below horizontal thus submerging the loop. Slowly remove the loop from the tube (approximately 10 seconds). Hold the lens in a vertical position with the freshly coated loop below the optic until dry. Repeat the dipping procedure with the other loop. Sterilize the lens with ethylene oxide and store at 4° C. until surgery.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

I claim:

1. A method for treating or preventing uveitis, comprising administering to a patient an anti-microtubule agent.

2. The method according to claim 1, wherein said anti-microtubule agent is paclitaxel, or an analogue or derivative thereof.

3. The method according to claim 1 wherein said anti-microtubule agent is camptothecin or a vinca alkaloid.

4. The method according to claim 1 wherein said anti-microtubule agent further comprises a polymer.

5. The method according to claim 1 wherein said anti-microtubule agent is released from an intraoccular lens or implant.

6. The method according to claim 1 wherein said anti-microtubule agent is administered systemically.

7. The method according to claim 1 wherein said anti-microtubule agent is administered by intraoccular or periocular injection.

8. The method according to claim 1 wherein said anti-microtubule agent is administered to the eye by eye drops.

9. A device, comprising an intraoccular lens which releases an anti-microtubule agent.

10. The device according to claim 9, wherein said lens is coated with an anti-microtubule agent.

11. The device according to claim 9 wherein said anti-microtubule agent is paclitaxel, or an analogue or derivative thereof.
